(19) United States
(12) Patent Application Publication
(10) Pub. No.: US 2025/0366514 A1
(43) Pub. Date: Dec. 4, 2025

(54) OXYGEN HOOKAH

(71) Applicant: Christian Jagodzinski, Miami Beach, FL (US)

(72) Inventor: Christian Jagodzinski, Miami Beach, FL (US)

(21) Appl. No.: 18/680,677

(22) Filed: May 31, 2024

(51) Int. Cl. A24F 1/30; A24F 40/05; A24F 40/485; A24F 40/60; A61M 16/10

(52) U.S. Cl. CPC A24F 1/30; A24F 40/05; A24F 40/485; A24F 40/60; A61M 16/1005

(57) ABSTRACT

An oxygen hookah having an oxygen cylinder, actuator, housing and tubing directing the oxygen from the cylinder and exiting the system to user through a mouthpiece.

### BACKGROUND OF THE INVENTION

Traditional hookahs that involve tobacco combustion and the inhalation of harmful substances. The risks associated with tobacco smoke, such as nicotine addiction and the inhalation of tar and carbon monoxide are well-known. There are many persons that would enjoy a non-tobacco based hookah experience. The present invention fills this void.

### SUMMARY OF THE INVENTION

In one embodiment, the present invention is an oxygen hookah constructed and arranged for a healthier experience.

The oxygen hookah of the present invention provides numerous and significantly novel improvements over traditional hookahs. These include, but are note limited to those enumerated below:

Flavored oxygen provides a clean and pure inhalation experience. It lacks the impurities found in tobacco and produces no residual odor, allowing users to enjoy the flavors without any lingering aftereffects. Flavored oxygen hookahs offer a wide range of flavors to choose from. Users can personalize their experience by selecting various flavors such as fruity, minty, or even exotic blends, catering to different tastes and preferences.

While traditional hookah sessions require the tobacco to heat up and the flavor to develop gradually, flavored oxygen hookah of the present invention provide an instantaneous effect. Users can experience the desired flavor immediately upon inhalation.

Flavored oxygen hookahs of the present invention do need coals or flames, thus eliminating the risk of burns and accidental fires. Additionally, since there is no tobacco combustion, there is no ash or residue to deal with, simplifying the cleaning process.

Flavored oxygen hookahs of the present invention offer a non-addictive experience since there is no nicotine present. This can be advantageous for those who enjoy the social aspect and relaxation of hookah but wish to avoid nicotine addiction.

Flavored oxygen of the present invention deliver more intense flavors compared to traditional hookahs. The absence of tobacco smoke allows users to fully experience and savor the chosen flavor without any interference.

By eliminating tobacco-related risks, flavored oxygen hookahs of the present invention reduce the potential for respiratory issues, cardiovascular problems, and other health concerns associated with tobacco smoking. It provides a safer option for those who want to enjoy hookah without compromising their well-being.

Traditional hookah sessions often involve the release of significant amounts of second-hand smoke, posing health risks to those around the smoker. Flavored oxygen hookahs of the present invention generate no smoke, ensuring a smoke-free environment for others while maintaining an enjoyable experience for the user.

Due to the absence of tobacco and associated health risks, flavored oxygen hookahs of the present invention are enjoyed in a wider range of social settings. It is more inclusive for non-smokers or individuals who are sensitive to tobacco smoke, allowing everyone to participate and enjoy the activity without concerns.

In one embodiment, the present invention is an oxygen hookah system comprising:
a water reservoir constructed and arranged as a base;
an oxygen container;
a hookah bowl;
at least one oxygen exit hose attached to said hookah bowl on a proximal end and having a hose exit on a distal end;
a circuit board;
a variable actuator operatively connected to said circuit board constructed and arranged on an outside surface of said system and constructed an arranged to control, in concert with said circuit board, oxygen flow from said oxygen container.

In one embodiment, there is tubing connecting said oxygen container gas output to said oxygen exit hose.

In one embodiment, the variable actuator is controlled by pressure differential imparted by a user inhaling through said mouthpiece.

In one embodiment, the variable actuator is configured for supplying between 1-100% oxygen to said hose exit.

In one embodiment, the present invention is an oxygen hookah system consisting of:
a water reservoir constructed and arranged as a base;
an oxygen container;
a hookah bowl;
at least one oxygen exit hose attached to said hookah bowl on a proximal end and having a hose exit on a distal end;
a circuit board;
a variable actuator operatively connected to said circuit board constructed and arranged on an outside surface of said system and constructed an arranged to control, in concert with said circuit board, oxygen flow from said oxygen container, wherein the tubing connecting said oxygen container gas output to said oxygen exit hose, wherein the variable actuator is controlled by pressure differential imparted by a user inhaling through said mouthpiece, and wherein the variable actuator is configured for supplying between 1-100% oxygen to said hose exit.

### BRIEF DESCRIPTION OF DRAWINGS

FIG. 1 is front perspective view according to one embodiments of the present invention.

FIG. 2 is a partial front view according to one embodiment of the present invention.

FIG. 3 is a front partial perspective view according to one embodiment of the present invention.

FIG. 4 is a front perspective view with the upper square e head module removed according to one embodiment of the present invention.

FIG. 5 is a front partial perspective view according to one embodiment of the present invention.

FIG. 6 is a plan view showing separated component parts according to one embodiment of the present invention.

FIG. 7 is a front cross-section view according to one embodiment of the present invention.

FIG. 8 is a front partial cross-section view according to one embodiment of the present invention.

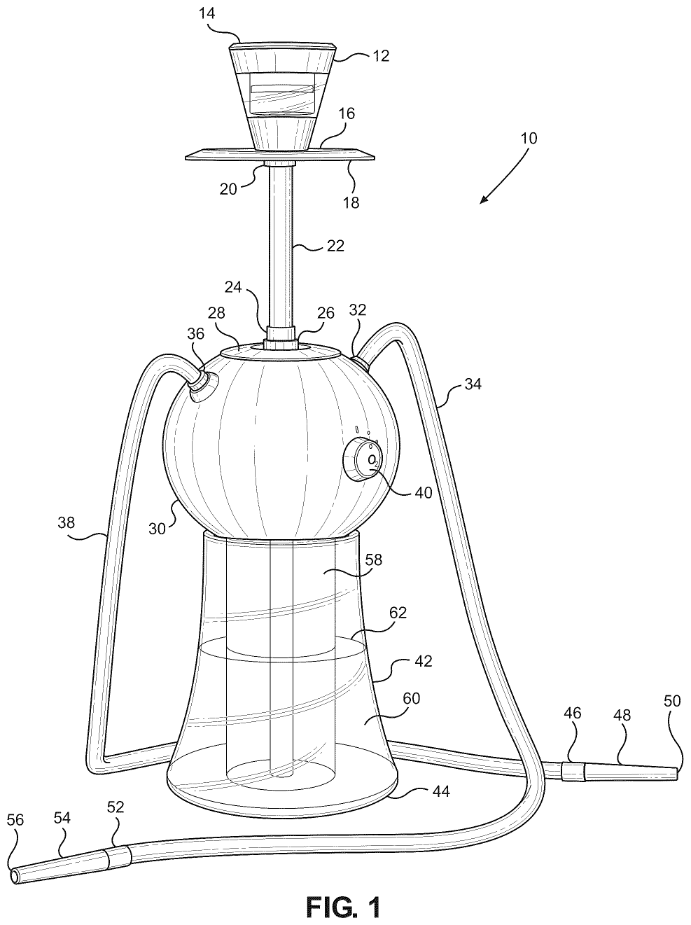

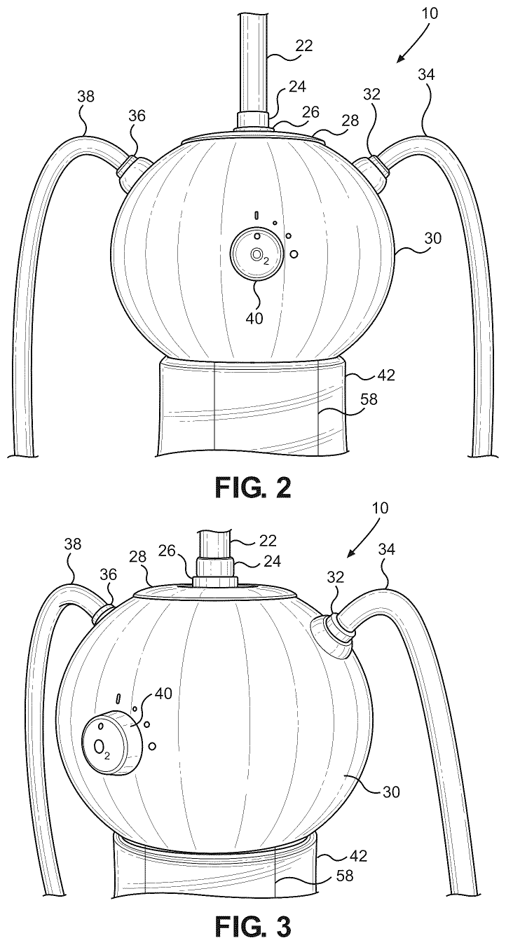

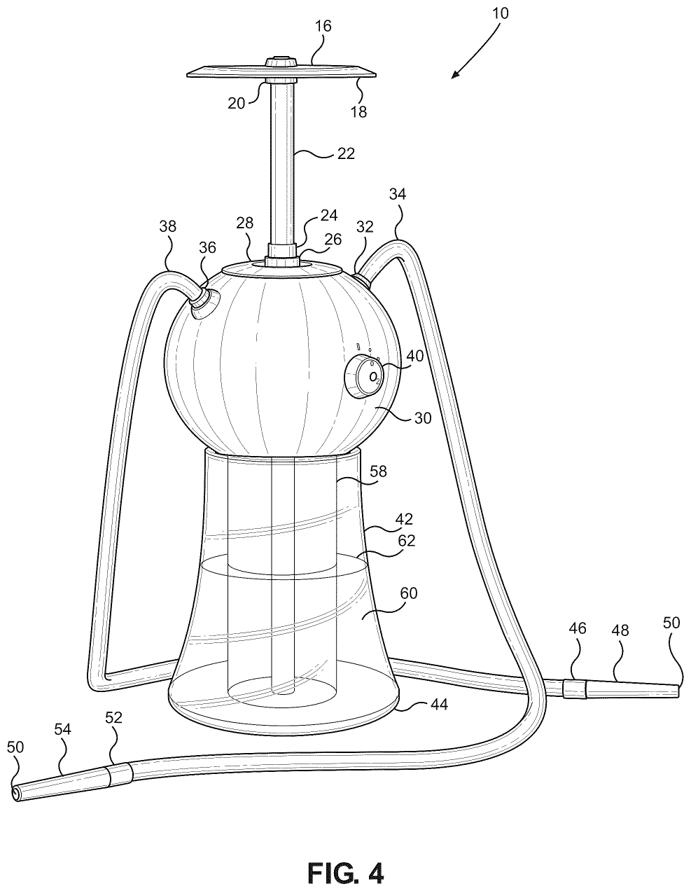

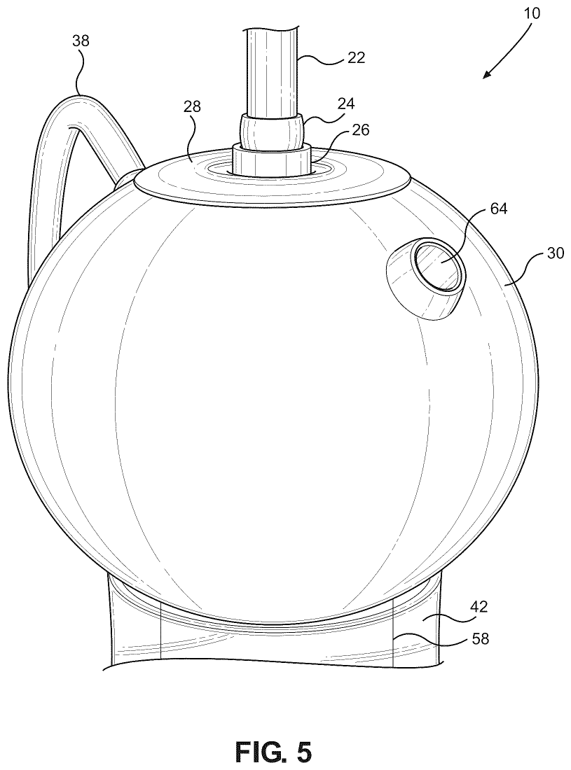

### DETAILED DESCRIPTION OF THE INVENTION

As generally understood from the accompanying figures, hookah system 10 includes water reservoir 42 having water reservoir 44 on its lower base. Water reservoir 42 is constructed arranged within inner cavity 60 that holds water 62 therein. Chamber 58 is within water reservoir 42. Hookah bowl 30 is positioned on top of water reservoir 42. Hookah 30 is constructed and arranged with control valve 40, first hose connector 32 with first hose 34 emanating therefrom having first hose mouthpiece 52 having first mouthpiece labial contact surface 54 and connected distal from hose connector 32 and including first hose exit 56 second hose connector 36 with second hose 38 emanating therefrom having second hose mouthpiece 46 with second house labial contact surface 48 and connected distal from hose connector 36 and including second hose exit 50. Top of bowl 30 is constructing and arranged with removable cover 28 having stem support 26 stem connector 24 supporting hookah stem 22. Attached to the top of stem 22 is connector 20 which supports ash catcher 16 held on with support 20 that contacts lower surface 18 of ash catcher 16, and square e head module 12 that has upper opening 14.

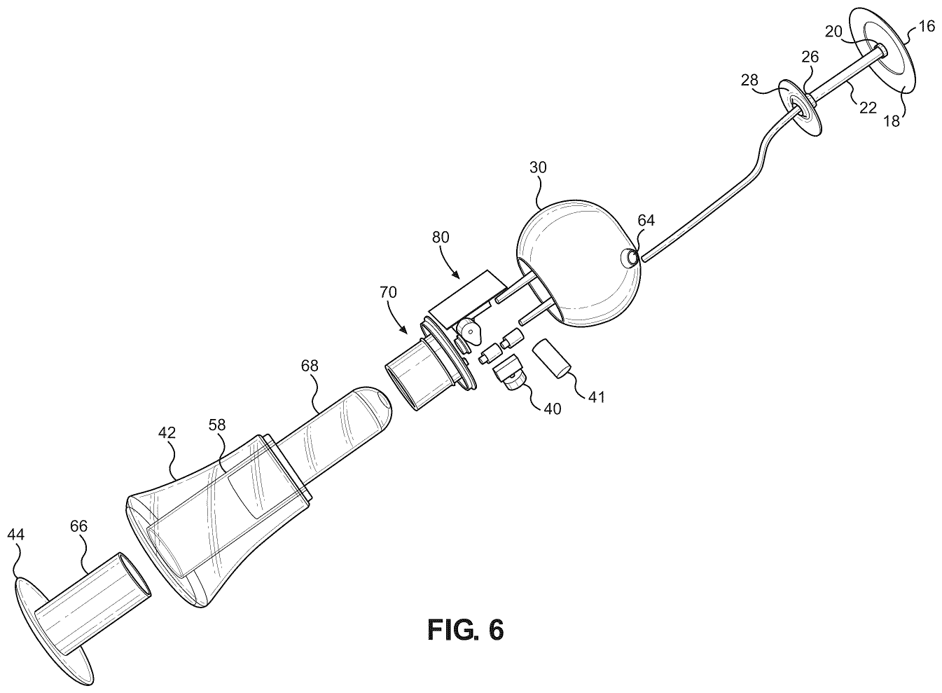

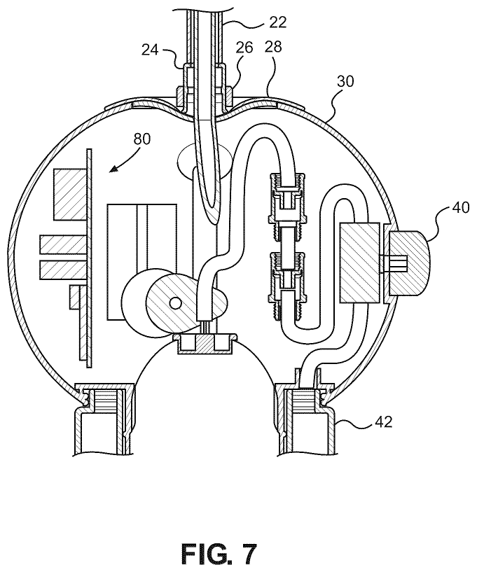

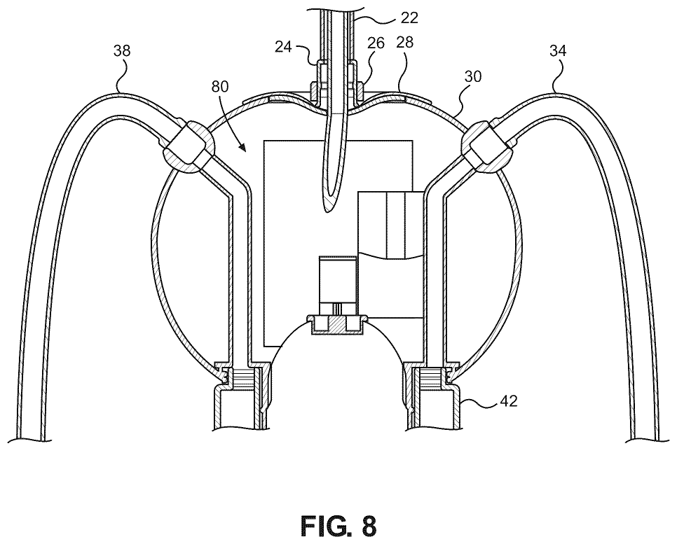

As demonstrated in FIGS. 6, 7, and 8, oxygen container 68 is nested within chamber 58 of water reservoir 42. Bottom cover 70 is removably attached to hookah bowl 30 and provides access to printed circuit board 80. Circuit board 80 contains electronics as known in the art to control the flow of oxygen from oxygen container 68 utilizing control valve 40. As generally understood, control valve 40, in one embodiment, is a variable actuator that when communication with circuit board 80 regulates the flow of oxygen.

Generally understood, oxygen container 68 is positioned within system 10 as demonstrated in the figures. When assembled, oxygen is directed towards either one or both of first hose 34 and second hose 38. Oxygen will eventually exit system 10 through either one or both of first to exit 56 or second hose exit 50.

In one embodiment, oxygen provided with an oxygen container 68 is flavored.

Control valve 40 is constructed and arranged to operate with the flow of oxygen emanating such that oxygen is metered and does not continually flow.

As further understood, system 10 includes configurations that provide for the look of a traditional hookah without providing the same functions. For example, there is no ash produced in the system of the present invention. Additionally, there is no need for any type of fire. There is no need for the water to cool a heated substrate but the water is present to provide the visual impression of a conventional hookah.

The mixture knob selects anything from “100% oxygen” to “100% filtered ambient air” and any combination thereof.

The actuator mechanism works with a motor as is known in the art. The impetus of the invention including release of oxygen whenever suction (pressure differential) on the inhalation pipe any mechanism.

Although the invention has been described with reference to specific embodiments and drawings, it is to be understood that the invention is not limited to those precise embodiments, and that various changes and modifications may be effected by one skilled in the art without departing from the scope or spirit of the present invention.

1. An oxygen hookah system comprising:
a water reservoir constructed and arranged as a base;
an oxygen container;
a hookah bowl;
at least one oxygen exit hose attached to said hookah bowl on a proximal end and having a hose exit on a distal end;
a circuit board;
a variable actuator operatively connected to said circuit board constructed and arranged on an outside surface of said system and constructed an arranged to control, in concert with said circuit board, oxygen flow from said oxygen container.

2. The system of claim 1 including tubing connecting said oxygen container gas output to said oxygen exit hose.

3. The system of claim 1 wherein said variable actuator is controlled by pressure differential imparted by a user inhaling through said mouthpiece.

4. The system of claim 1 wherein said variable actuator is configured for supplying between 1-100% oxygen to said hose exit.
